(19) United States
(12) Patent Application Publication
(10) Pub. No.: US 2005/0228284 A1
(43) Pub. Date: Oct. 13, 2005

(54) System and method for power management in an ultrasound system

(76) Inventors: Charles Edward Baumgartner, Niskayuna, NY (US); George Charles Sogoian, Glenville, NY (US); Robert Stephen Lewandowski, Amsterdam, NY (US)

Correspondence Address: Tait R. Swanson;Fletcher Yoder, P.O. Box 692289, Houston, TX, 77269-2289, US

(21) Appl. No.: 10/814,830

(22) Filed: Mar. 31, 2004

(51) Int. Cl. A61B 8/00

(52) U.S. Cl. 600/459

(57) ABSTRACT

An ultrasound system including an ultrasound probe, which has an ultrasonic transducer and a physical sensor adapted to sense engagement with a subject to be scanned by the ultrasonic transducer. The ultrasound system also includes a control system coupled to the ultrasound probe.

### BACKGROUND

The invention relates generally to imaging systems and, more specifically, to a method and apparatus for controlling power and heat generated by medical ultrasound imaging probes based on sensed contact with a subject.

Ultrasound imaging technology is commonly used in several applications, such as detecting tumors, lesions and cysts, assessing functions of the heart, monitoring development of the fetus, and diagnosing malignancies of a number of organs. In many of these imaging applications, a clinician holds the ultrasound probe to analyze a patient's condition. The current trend is to position many of the electronic components of the ultrasound system into the ultrasound probe. In certain applications, this repositioning of components may expedite the imaging process or add more features to the ultrasound probe. For example, increasing the number of transducer rows in the elevation direction allows for multi-focal depths improving image quality and/or reducing imaging time. Further advances in imaging can be obtained with a two-dimensional transducer array that possesses a large number of transducer elements in both the elevation and azimuthal directions. Connection of these multi-row arrays to a conventional ultrasound system however requires a large number of cable connections as each transducer element is independently connected to the system. Reduction in the number of cable connections is obtained by relocating parts of the ultrasound system such as multiplexers, transmit/receive switches, beamformers, or pulsers into the handle. Unfortunately, the increased number of components in the ultrasound probe generally increases the power levels and heat generation within the ultrasound probe.

In certain applications, heat sinks are used in the ultrasound probe to cool these components and the ultrasound probe. Thermally conductive epoxy materials or fluid coolants also may be employed to facilitate cooling. In addition, thermally conductive materials may be disposed in the handle of the ultrasound probe to conduct and radiate heat out of the ultrasound probe. Unfortunately, these heat transfer techniques are insufficient to cool the ultrasound probe if too many electronic components are operating within the ultrasound probe. However, the use of added components for active cooling of the ultrasound probe adds cost and complexity to the probe.

In other applications, the power of the ultrasound probe has been switched to a low power mode after a predetermined time or upon exceeding a certain temperature. Previous applications sense the ultrasound pulse/echo to determine when the probe is actively imaging as a means of reducing heat generated by an ultrasound probe. However, such a technique could interrupt the imaging process.

Therefore, a need exists for a technique to facilitate cooling and power control of an ultrasound imaging system.

### BRIEF DESCRIPTION

An ultrasound system including an ultrasound probe, which has an ultrasonic transducer and a physical sensor adapted to sense engagement with a subject to be scanned by the ultrasonic transducer. The ultrasound system also includes a control system coupled to the ultrasound probe. Also included is a method for controlling heat in an ultrasound system. The method involves sensing physical engagement of an ultrasound module with a subject, and, switching power modes of the ultrasound module based on the sensed engagement.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatic view of an ultrasound system in accordance with embodiments of the present technique;

FIG. 2 is a diagrammatic view of an exemplary ultrasound probe in accordance with embodiments of the present technique; and

FIG. 3 is a flow chart in accordance with one aspect of the present technique.

### DETAILED DESCRIPTION

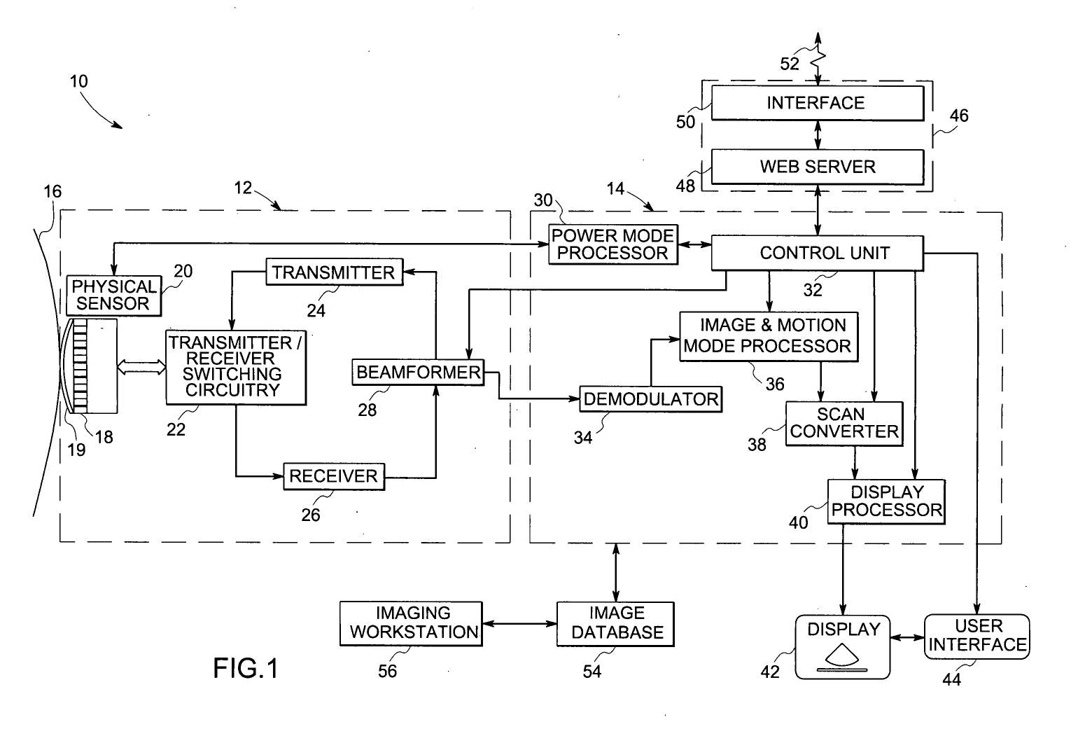

FIG. 1 is a diagrammatic view of a typical ultrasound system in accordance with embodiments of the present technique. The illustrated ultrasound system 10 comprises an ultrasound probe 12 and an ultrasound control unit or base console 14. For example, the ultrasound probe 12 may comprise a handheld or generally portable ultrasound probe, which a clinician or other user can operate to scan a subject. By further example, the ultrasound control unit 14 may comprise a main ultrasound system, which includes a variety of stationary and/or movable devices to control and process ultrasounds scans obtained by the ultrasound probe 12.

The ultrasound probe 12 comprises a piezoelectric transducer array 18, a front face or acoustic lens structure 19, one or more physical sensors 20, transmitter/receiver switching circuitry 22, a transmitter 24, a receiver 26, and a beamformer 28. In addition, various other electronic components, such as those typically disposed in the ultrasound control unit 14, may be incorporated into the ultrasound probe 12. Alternatively, in certain embodiments, one or more of the switching circuitry 22, the transmitter 24, and the beamformer 28 are located within the ultrasound control unit 14, rather than the ultrasound probe 12. Thus, embodiments of the ultrasound probe 12 have primarily the transducer array 18 and the physical sensors 20. As discussed in detail below, the physical sensors 20 facilitate power management of the ultrasound probe 12 by sensing physical engagement or proximity of a subject, such as a patient, such that the ultrasound probe 12 can be powered up for ultrasonic scanning.

Ultrasound control unit 14 comprises a power mode processor 30, control unit 32, a demodulator 34, an image and motion mode processor 36, a scan converter 38, and a display processor 40. The display processor 40 is further coupled to a display unit 42 for displaying images. User interface unit 44 is in communicative coupling with the control unit 32 and the display unit 42. The control unit 32 may also be coupled to a remote connectivity module 46 comprising a web server 48 and a remote connectivity interface 50 for coupling the ultrasound system to a network, via link 52. Ultrasound control unit 14 may be further coupled to an image database 54 to receive ultrasound image data. In turn, the image database may be in communicative coupling with imaging workstation 56.

The architectures and modules of system 10 may comprise a variety of hardware and software components. For example, the system may include hardware components, such as circuit boards with digital signal processors. Also, the system 10 can have computer readable instructions executable on a variety of media, storage devices, or hardware, such as personal computer.

In the ultrasound probe 12, the physical sensors 20 detect when the ultrasound probe 12 is in contact, close proximity, or generally approaching the subject to be ultrasonically scanned by the ultrasonic transducer array 18. For example, the physical sensors 20 may sense temperature, pressure, distance, or other physical characteristics of the subject 16, such that the ultrasound probe 12 can be powered up from a low power mode to an operational power mode for performing an ultrasonic scan upon engaging the subject 16. The transducer array 18 is adapted to contact with subject 16, such that an ultrasonic scan may be performed to analyze internal features of the subject 16.

The transducer array 18 is coupled to the transmitter/receiver switching circuitry 22, which is further coupled to the pulser or transmitter 24 which sends electrical signals to the transducer elements and the receiver 26 which receives electrical signals generated by the transducer from the returning ultrasound echo. Typically the same transducer elements both generate and receive ultrasound energy in a pulse-echo mode, although different elements on the transducer may be used for these functions in selected cases. The transmitter/receiver switching circuitry 22 switches the transmitter 24 and receiver 26, such that the switching is multiplexed in time. In operation, the transmitter 24 is powered when ultrasound energy has to be transmitted into the body of the subject 16, and the transmitter/receiver switching circuitry 22 powers the receiver circuitry 26 when the transducer array 18 receives the echo signals from the tissue layers of the subject 16. Beamformer 28 is coupled to transmitter 24, receiver 26, control unit 32, and demodulator 34. The operation of the beamformer will be explained in detail below.

In ultrasound control unit 14, the control unit 32 is coupled to a power mode processor 30, which is communicatively coupled to physical sensors 20. Similarly, the power mode processor 30 may be located within the ultrasound probe 12. As discussed above, the physical sensors 20 detect various physical characteristics, such as heat transfer, contact pressure, proximity distance, and other characteristics of the subject 16. These sensed physical characteristics are then transmitted to the power mode processor 30, which controls the power mode of the ultrasound probe 12 via the control unit 32. For example, the power mode processor 30 and control unit 32 may comprise a plurality of power modes, such as a low power mode and an operational power mode, for the ultrasound probe 12. If the physical sensors 20 detect engagement or approaching engagement of the ultrasound probe 12 with the subject 16, then the power mode may be shifted from the low power mode to the operational power mode to facilitate ultrasonic scanning of the subject 16. Similarly, if the physical sensors 20 detect disengagement of the ultrasound probe 12 away from the subject 16, then the power mode may be shifted from the operational power mode to the low power mode to facilitate power savings and cooler operation of the ultrasound probe 12 while scanning is not being performed. Accordingly, the control unit 32 modulates the power of the electronic components within the ultrasound probe 12 in accordance with the signals received from the power mode processor 30 and the physical sensors 20.

In addition, the ultrasound control unit 14 has the demodulator 34, which is coupled to the beamformer 28 of the ultrasound probe 12. In turn, the demodulator 34 is coupled to the image and motion mode processor 36. Control unit 32 drives the image and motion mode processor 36, scan converter 38, display processor 40, and the user interface unit 44. The image and motion mode processor 36 is also coupled to the scan converter 38. The scan converter 38 sends image data to the display processor 40, which drives the display unit 42.

In operation of ultrasound system 10, the ultrasound probe 12 transmits ultrasound energy from the ultrasonic transducers 18 into subject 16. The ultrasound probe 12 then receives and processes backscattered ultrasound signals from the subject 16 to process, create, and display an image. An ultrasound energy beam of a desired shape originating at the surface of the transducer array 18 at a desired steering angle is generated by the beamformer 28. The beam of ultrasound energy is formed in the subject 16 within a scan plane along a scan line when the transducer array 18 is acoustically coupled to the subject 16 by using, for example, ultrasound gel.

The illustrated transducer array 18 comprises a two-way transducer. When ultrasound waves are transmitted into a subject 16, the ultrasound waves are backscattered off the tissue and blood within the subject 16. The transducer array 18 receives the backscattered waves at different times, depending on the distance into the tissue they return from, and the angle with respect to the surface of the transducer array 18 at which they return. The transducer elements are responsive to the backscattered waves and convert the ultrasound energy from the backscattered waves into electrical signals.

The electrical signals received by the transducer array 18 are routed through the transmitter/receiver switching circuitry 22 to the receiver 26. The illustrated receiver 26 amplifies and digitizes the received signals after proper gain compensation. The digitized signals correspond to the backscattered waves received by each transducer element at various times. After digitization, the signals still preserve the amplitude and phase information of the backscattered waves.

The digitized received signals are transmitted to beamformer 28, along with control signals generated by the control unit 32. Beamformer 28 utilizes the control signals to form a beam originating from a point on the surface of transducer array 18 at a steering angle typically corresponding to the point and steering angle of the ultrasound beam previously transmitted along the scan line. The beamformer 28 processes the received signals by performing time delaying and focusing, in accordance with the control signals from the control unit, to create return beam signals corresponding to sample volumes along a scan line in the scan plane within the subject 16. Thus, phase, amplitude, and timing information of the received signals from the various transducer elements is used to process the signals.

In turn, the processed signals are sent to ultrasound control unit 14. Demodulator 34 demodulates the signals and transmits the demodulated signals to the image and motion mode processor 36. Image and motion mode processor 36 and scan converter 38 utilize the demodulated signals to create pixel data in the display format. The pixel data is sent to display processor 40 to perform any final spatial or temporal filtering, to apply grayscale or color to the pixel data, and to convert the digital pixel data to analog data for display on display unit 42. The user interface unit 44 is coupled to the control unit 32, which controls the data displayed on display unit 42.

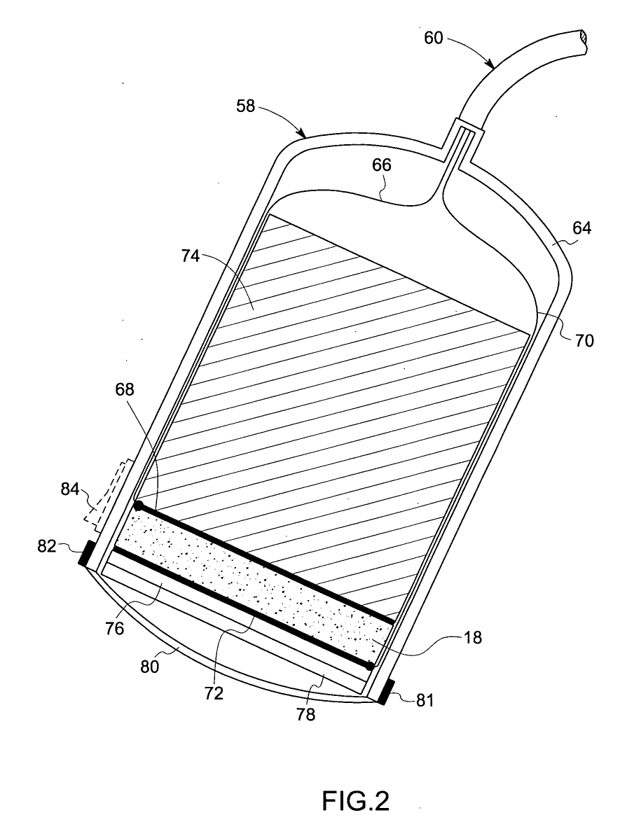

FIG. 2 is a cross-sectional top view of an exemplary ultrasound probe 58 in accordance with embodiments of the present technique. Although the ultrasound probe 58 may have any number of transducer elements, FIG. 2 illustrates a cross-section of a single transducer element of a transducer array 18. Depending on the particular embodiment of the ultrasound probe 58, the transducer array 18 may comprise multiple rows of elements or a two-dimensional configuration comprising any suitable number of elements, e.g., several thousand elements. Moreover, the transducer array 18 may comprise a variety of materials, structures, and configurations. For example, the illustrated transducer array 18 may comprise one or more lead zirconium transducers, which are referred to as PZT transducers. In certain embodiments, the ultrasound probe 58 has a linear array of the PZT transducers, such as a linear array of 128 sensing elements.

As further illustrated in FIG. 2, the ultrasound probe 58 includes a cable 60, a housing 64, a live wire 66, a live electrode 68, a ground wire 70, a ground electrode 72, backing material 74, acoustic matching layers 76 and 78, a front face or acoustic lens 80, a temperature sensing element 81, a pressure sensing element 82, and an optional manual power switch 84, as discussed in further detail below. The cable 60 connects the ultrasound probe 58 with an ultrasound console, e.g., ultrasound control unit 14. Although not illustrated in FIG. 2, the ultrasound probe 58 also may include a variety of additional components in the housing 64, as described above with reference to FIG. 1. The illustrated transducer array 18 is disposed between a pair of electrodes, i.e., the live electrode 68 and the ground electrode 72. The electrodes 68 and 72 are coupled to the ultrasound console 14 by the live wire 66 and the ground wire 70, respectively. The acoustic matching layers 76 and 78 enable the ultrasound energy emitted from the transducer array 18 (and transmitter 24 of FIG. 1) to pass to the face or acoustic lens 80 of the ultrasound probe 58 with relatively minimal reflection back into the transducer array 18. The acoustic lens 80 both protects the surface of the ultrasound probe 58 and focuses the emitted ultrasound energy to a pre-selected focal depth into the body of the subject 16. The backing material 74 absorbs any ultrasound energy generated by the transducer array 18 in the direction away from the face or acoustic lens 80 of the ultrasound probe 58.

In the illustrated embodiment, the temperature sensing element 81 and the pressure sensing element 82 are disposed at outer peripheral portions of the front face or lens 80 of the ultrasound probe 58. For example, the sensing elements 81 and 82 may be embedded into the lens 80 or located next to the lens 80. Although not illustrated, other embodiments may include various other types of physical sensors, such as distance or proximity sensors, motion sensors, and so forth. As discussed above, these physical sensors, e.g., 81 and 82, facilitate detection of the subject 16, such that the ultrasound system 10 can power up or increase power modes when the ultrasound probe 58 is in a position to begin ultrasound scanning. Similarly, the physical sensors enable powering down or decreasing power modes when the ultrasound probe 58 is out of a position to perform ultrasound scanning. In accordance with one aspect of the embodiment, the manual power switch 84 is also present on the housing 64. For example, this manual power switch 84 may enable a user to change power modes of the ultrasound probe 58 manually upon positioning in engagement or disengagement with the subject 16.

The temperature sensing element 81 senses any heat transfer between the ultrasound probe 58 and the subject 16. When the ultrasound probe 58 is in contact with the body of the subject 16, the temperature sensing element 81 senses the heat transferred from the ultrasound probe 58 to the subject 16, or vice-versa, because the ultrasound probe 58 may either be at a lower temperature or at a higher temperature than the subject 16. However, the temperature sensing element 81 may comprise a variety of sensing techniques to detect thermal engagement, disengagement, or general proximity with the subject 16. In turn, the sensed signal is transmitted to the power mode processor 30 to be processed, as discussed above with reference to FIG. 1. The power mode processor 30 sends the processed signals of the temperature sensing and the power mode data to the control unit 32. The control unit 32 switches the electronic components within the ultrasound probe 58 to a low power mode when the ultrasound probe 58 is away from the subject 16, i.e., in contact with air or other non-absorbing media, rather than the body of the subject 16. The ultrasound probe 58 may additionally comprise a physiological sensing element, which senses the presence of skin or other physiological characteristics of the subject 16, so that the electronic components inside the ultrasound probe 58 may be switched “ON” when the ultrasound probe 58 is in contact or proximity with the subject 16. Further, in other embodiments, the control unit 32 may switch the ultrasound probe 58 to a plurality of low, medium, high or other power modes during operation to save power, and avoid undesirable heating of the ultrasound probe.

Similarly, the pressure sensing element 82 detects modes of the ultrasound probe 58 by either sensing contact pressure of the probe 58 with the subject 16 or by sensing pressure of a user grasping the probe 58 in preparation of performing an ultrasonic scan of the subject 16. For example, when the ultrasound probe 58 is held in hand for monitoring the subject 16, and the ultrasound probe 58 is switched “ON”, the scanning process is also switched into a high power mode. However, there may be cases when the ultrasound probe 58 is switched “ON” and is not scanning the subject 16. For example, in cases when the operator is in the middle of scanning a subject 16 and wishes to study the images taken, he may place the ultrasound probe 58 in an ultrasound probe holder, with the ultrasound probe still in the “ON” state. In such a case, the pressure sensing element 82 may transmit a signal to the power mode processor 30 that the ultrasound probe 58 is currently not being held in hand for scanning or pressed against the subject 16. Based on the signal, the power mode processor 30 sends the processed signals of the pressure sensing and the power mode data to the control unit 32, which then switches the electronic components of the ultrasound probe 58 into a low power mode.

In the above cases, based on the signals received from the temperature sensing element 81 or the pressure sensing element 82, the control unit 32 switches the ultrasound probe 58 into a plurality of power modes. The power mode processor 30 processes the signals received from the temperature sensing element 81 and/or the pressure sensing element 82 prior to sending it to the control unit 32. It should be noted that the control unit 32 switches the ultrasound probe 58 into a high power mode based on a heat transfer detected between the subject 16 and the ultrasound probe 58, and/or pressure detection of the ultrasound probe 58 being held in hand for scanning. It will be appreciated by those skilled in the art that the power mode processor may be a dedicated processor such as an ASIC (application specific integrated circuit) or, a digital signal processor configured for processing the signals. Alternatively, computer readable instructions may be embedded in the processor of the control unit 32 to process the above signals.

In another embodiment, the ultrasound probe 58 comprises the manual power switch 84 for switching the ultrasound probe 58 between power modes at the probe 58 itself, rather than at the ultrasound control unit 14 (see FIG. 1). The operator can utilize this switch 84 to toggle the ultrasound probe manually between “ON” or “OFF” positions. The switch 84 may be utilized in addition to the sensing elements 81 and 82, or may be utilized alone to provide power and heat control of the ultrasound probe 58.

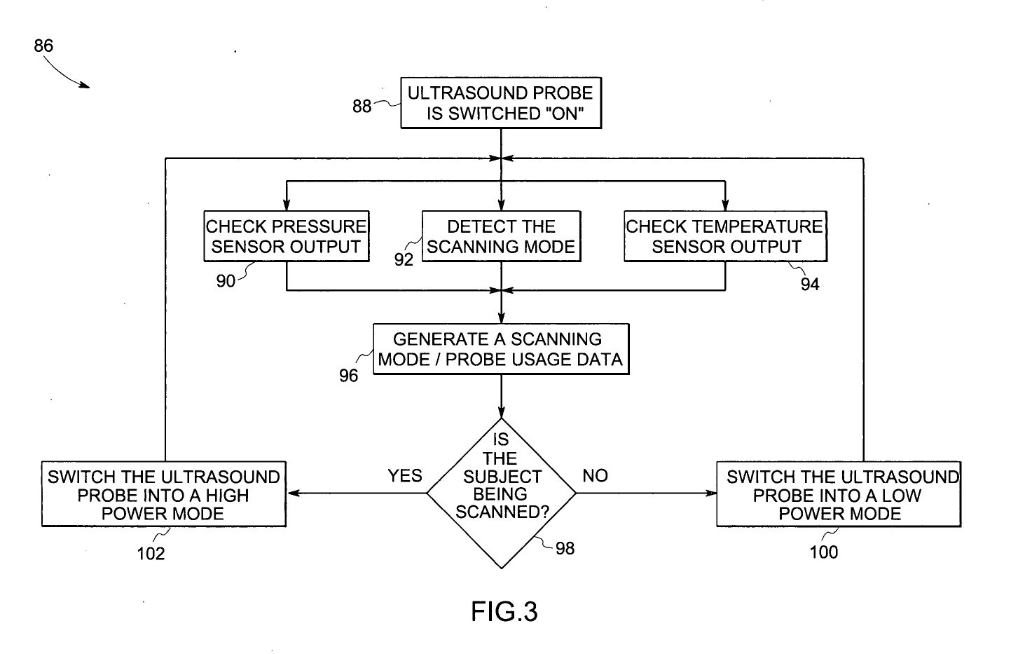

Referring to FIG. 3, a flow chart 86 illustrates one technique for controlling heat generated in an ultrasound system. At block 88, the process 86 begins with the ultrasound probe 58 being switched “ON” manually. Whether the ultrasound probe 58 is actively scanning a subject can be determined in any number of ways, which include a pressure sensing method 90, a temperature sensing method 94, and another physical sensing method 92. As discussed above with reference to FIG. 2, the contact pressure of the operator using the ultrasound probe can be detected by the pressure sensing element 82. The contact pressure is indicative of the ultrasound probe 58 being held in hand or engaged against the subject 16 in preparation for ultrasonic scanning. Similarly, the temperature sensing element 81 detects the contact of the ultrasound probe 58 with the body of the subject 16 for active ultrasonic scanning. Since the ultrasound probe 58 is engaged with the subject 16 only for ultrasonic scanning, the heat transfer is indicative of a desire to perform ultrasound scanning.

At block 96, the sensing elements 90, 92, and 94 generate and transmit data indicative of the ultrasound probe 58 usage to the control unit 32. In certain embodiments, the control unit 32 is located within the ultrasound probe 58, rather than the main system (e.g., ultrasound control unit 14). The processor receives the data and processes it to generate control signals for switching the ultrasound probe 58 into a plurality of different power modes 98. When the ultrasound probe is switched “ON” and the operator is scanning the subject, the ultrasound probe is switched into a normal full power mode 102. Once the control unit 32 detects that the ultrasound probe 58 is in a power “ON” state but is not actively scanning the subject 16, the ultrasound probe 58 may be switched into several low power modes 100. Depending on the usage of the ultrasound probe 58, the control unit 32 switches the ultrasound probe 58 into one of the several low power modes. When the operator resumes scanning, the control unit 32 switches the ultrasound probe 58 into the normal full power mode.

While only certain features of the invention have been illustrated and described herein, many modifications and changes will occur to those skilled in the art. It is, therefore, to be understood that the appended claims are intended to cover all such modifications and changes that fall within the true spirit of the invention.

1. An ultrasound system, comprising:
an ultrasound probe, comprising
an ultrasonic transducer; and
a physical sensor adapted to sense engagement with a subject to be scanned by the ultrasonic transducer; and
a control system coupled to the ultrasound probe.

2. The system of claim 1, wherein the control system is configured to control power modes of the ultrasound probe based on feedback from the physical sensor.

3. The system of claim 1, wherein the ultrasound probe comprises a hand holdable body.

4. The system of claim 3, wherein the hand holdable body comprises at least a portion of a beamformer.

5. The system of claim 1, wherein the physical sensor comprises a pressure sensor configured to detect a contact pressure with the subject.

6. The system of claim 5, wherein the pressure sensor comprises a piezoelectric sensor element.

7. The system of claim 1, wherein the physical sensor comprises a temperature sensor configured to detect thermal proximity with the subject.

8. The system of claim 1, wherein the physical sensor comprises a manual power switch.

9. A method for controlling heat in an ultrasound system, the method comprising:
physically sensing engagement of an ultrasound module with a subject; and
switching power modes of the ultrasound module based on the sensed engagement.

10. The method of claim 9, comprising:
manually switching the power modes at a handheld unit of the ultrasound module.

11. The method of claim 9, wherein physically sensing engagement comprises detecting a contact pressure with the subject.

12. The method of claim 9, wherein physically sensing engagement comprises detecting thermal proximity of the subject.

13. The method of claim 9, wherein physically sensing engagement comprises detecting physical contact of a hand holdable probe of the ultrasound module with the subject.

14. The method of claim 9, wherein switching power modes comprises increasing power of the ultrasound module upon sensing engagement with the subject to enable ultrasonic scanning of the subject.

15. An ultrasound system, comprising:
a hand holdable ultrasound probe, comprising:
an ultrasonic transducer configured to scan a subject; and
a sensing element configured to detect physical proximity of the hand holdable ultrasound probe relative to the subject; and
a control system coupled to the hand holdable ultrasound probe, wherein the control system is configured to switch the ultrasound probe between a plurality of power modes based on feedback from the sensing element.

16. The system of claim 15, wherein the sensing element comprises a pressure sensor configured to detect a contact pressure between the hand holdable ultrasound probe and the subject.

17. The system of claim 15, wherein the sensing element comprises a temperature sensor configured to detect a temperature differential between the hand holdable ultrasound probe and the subject.

18. A method of manufacture, comprising:
providing an ultrasound unit having an ultrasound transducer to scan a subject and a physical sensor to detect proximity of a subject relative to the ultrasound unit.

19. The method of claim 18, wherein providing the ultrasound unit comprises providing a hand holdable body having the ultrasound transducer, the physical sensor, and a beamformer coupled to the ultrasound transducer.

20. The method of claim 18, wherein providing the ultrasound unit comprises disposing a pressure sensor on the hand holdable body of the ultrasound unit.

21. The method of claim 18, wherein providing the ultrasound unit comprises disposing a temperature sensor on the hand holdable body of the ultrasound unit.

22. The method of claim 18, comprising providing a control system to change power levels of the ultrasound unit based on the feedback from the physical sensor.

23. An ultrasound system, comprising:
means for physically detecting proximity of an ultrasound module relative to a subject to be scanned by ultrasonic transducers of the ultrasound module; and
means for switching power modes the ultrasound probe based on proximity feedback from the means for physically detecting.
